(19) United States
(12) Patent Application Publication
(10) Pub. No.: US 2011/0318287 A1
(43) Pub. Date: Dec. 29, 2011

(54) ANTI-FUNGAL NAIL POLISH

(75) Inventor: Gary S. Evans, Closter, NJ (US)

(73) Assignee: ALDE ASSOCIATES, LLC, Closter, NJ (US)

(21) Appl. No.: 13/167,984

(22) Filed: Jun. 24, 2011

Related U.S. Application Data
(60) Provisional application No. 61/358,517, filed on Jun. 25, 2010.

(51) Int. Cl. A61K 31/20; A61P 31/10

(52) U.S. Cl. 424/61

(57) ABSTRACT

A premium, colored, acrylic nail polish or lacquer combined with at least one FDA-approved antifungal agent for the purpose of providing an attractive nail coating with antifungal properties.

### CROSS REFERENCE TO RELATED APPLICATION

This application claims the benefit of U.S. Provisional Application No. 61/358,517, filed Jun. 25, 2010, the disclosure of which is incorporated herein in its entirety by reference thereto.

### FIELD OF THE INVENTION

The present invention relates in general to nail polishes and in particular to antifungal nail polishes.

### BACKGROUND OF THE INVENTION

A fungal nail infection occurs when a fungus attacks a fingernail, toenail, or nail bed. Individuals who frequent public gyms, swimming pools or showers are more susceptible to contracting an infection. Likewise, individuals who receive nail services, such as a manicure or pedicure, performed with unsterilized tools place themselves at a greater risk for contracting a fungal or other infection.

Fungal nail infections can be painful, unattractive and difficult to treat. In some cases, antifungal pills must be prescribed. However, these pills can have severe side effects.

Infections can be treated using undecylenic acid, a United States Food and Drug Administration (FDA) approved over-the-counter fungicidal organic fatty acid commonly available in a cream or liquid solution form. Heretofore, in order for undecylenic acid to be effective as an antifungal agent, all nail polish must be removed.

Currently, several nail polish preparations are available that contain the active ingredient Melaleuca alternifolia (also known as tea tree oil). However, there is a lack of definitive evidence that this ingredient may be effective in treating fungal infections.

An advantage exists, therefore, for a cosmetic product that will help prevent and/or treat fungus infection without the risk of side effects.

### SUMMARY OF THE INVENTION

The present invention provides for a premium, colored, acrylic nail polish or lacquer combined with one or more FDA-approved antifungal agents for the purpose of providing an attractive nail coating with antifungal properties.

### DETAILED DESCRIPTION OF THE INVENTION

The present invention relates to a non-penetrating, colored, acrylic nail polish or lacquer combined with one or more FDA-approved antifungal agents for the purpose of providing an attractive nail coating with antifungal properties.

The preferred embodiment utilizes the ratio of about nine parts colored lacquer nail polish to about one part undecylenic acid, i.e., about 10% undecylenic acid to about 90% lacquer, by volume. However, depending upon the presence or absence of pigment and the type of pigment selected for the end user application, it is possible that less than about 10% of antifungal agent may be used in the composition with a corresponding increase in volume of lacquer. Further, it will be understood that any FDA-approved antifungal agents may be used in the composition according to the invention, which agents have been proven effective in treating the organisms that cause nail fungus, onychomycosis. For example, the composition may include one or more other FDA-approved antifungal agents as active antifungal ingredient(s) of the formulation in addition to, in lieu of, or in combination with, undecylenic acid.

The color or pigment may be any color whereby the therapeutic nail polish according to the invention looks and feels like conventional nail polish. Simultaneously, the nail polish provides an effective antifungal treatment which may be used to treat existing or prevent future fungal nail infections.

Significantly, the preferred composition is non-penetrating. As such, the antifungal agent remains on top of the nail and around the margins thereof whereby it prevents passage of fungus into the nail in the first instance while also forming a preventive barrier between the nail and pre-existing fungal infections.

Variations can be made therein by those skilled in the art without departing from the spirit and scope of the invention as claimed herein.

What is claimed is:

1. An antifungal nail polish comprising no greater than about 10% of at least one FDA-approved antifungal agent to no less than about 90% lacquer by volume.

2. The nail polish of claim 1 wherein said at least one FDA-approved antifungal agent includes undecylenic acid.

3. The nail polish of claim 1 wherein the polish is colored.

4. The nail polish of claim 1 wherein the polish is non-penetrating.
